(19) United States
(12) Patent Application Publication
(10) Pub. No.: US 2026/0138935 A1
(43) Pub. Date: May 21, 2026

(54) System and method for the production of chemical compounds and nutrient optimized fertilizer from animal waste products

(71) Applicants: Daniel Deocampo, Avondale Estates, GA (US); Richard Jensen, Atlanta, GA (US)

(72) Inventors: Daniel Deocampo, Avondale Estates, GA (US); Richard Jensen, Atlanta, GA (US)

(73) Assignee: Nalari Scientific, LLC, Atlanta, GA (US)

(21) Appl. No.: 18/953,427

(22) Filed: Nov. 20, 2024

(51) Int. Cl. C05F 3/00; C07D 233/88; C08H 8/00

(52) U.S. Cl. CPC C05F 3/00; C07D 233/88; C08H 8/00

(57) ABSTRACT

The present invention relates to the beneficiation of animal waste products, such as chicken manure and chicken litter, using sequential solubility extractions to separate the various components of the animal waste product. The separated products are then further processed to produce various value-added end products, such as allantoin and other uric acid derivatives, along with other chemicals and derivatives of the lignocellulosic biomass and mineral salts contained within the animal waste product.

### CROSS-REFERENCE TO RELATED APPLICATIONS

This application claims the benefit of priority to U.S. Provisional Application No. 63/600,818 filed Nov. 20, 2023, which is incorporated herein by reference.

### FIELD

The invention relates to the beneficiation of animal waste products and chemical transformation of substances contained therein.

### BACKGROUND

Nitrogen-rich organic compounds are important chemical components of many biological, environmental, medical, and industrial systems. They are therefore crucial components of the global economy. Many nitrogenous compounds are found in nature, but most such chemicals used in modern industry are synthesized from raw materials originating as petroleum or other carbonaceous fossil deposits. In most cases, the nitrogen used for such synthesis is fixed as ammonia using the Haber-Bosch method of ammonia generation, which remains the world's primary means of nitrogen fixation despite the very high energy cost.

Demand for nitrogenous organic compounds continues to grow as new chemicals are developed, as well as with the growth of established chemical markets. One example of an established and growing market is that of allantoin (C₄H₆N₄O₃), a widely-used ingredient in skin care products and dermatological pharmaceuticals with a global market approaching $1 billion. Allantoin is a heterocyclic organic compound rich in nitrogen, that is important in many organisms for nitrogen cycling, and an important intermediate in the catabolic degradation of purines, important for protein processing in most organisms. It is found naturally concentrated in a number of organisms such as legumes, comfrey, and certain lichen (Xu et al., 2011), but not generally at industrially useful levels. Most allantoin for industrial, commercial, or pharmaceutical use is synthesized through the reaction of glyoxalic acid with urea. Glyoxalic acid, in turn, is largely derived through chemical synthesis from petroleum derivatives, whereas the amine components of industrial urea generally originate from Haber-Bosch nitrogen fixation. With a growing global desire to decarbonize industrial manufacturing and rely on more sustainable processes with a smaller climate impact, there is a demand for innovation with the potential to disrupt such existing chemical markets.

One example of a new chemical that is transforming industry while creating demand for innovative manufacture of nitrogenous organic compounds is the family of compounds known as “ionic liquids” (IL's). IL's are ionically bonded compounds, composed of a cation and an anion, that are liquids at room temperature. Hence they are also known as “liquid salts” or “molten salts”. A great diversity of IL's is possible, because many different cations may be matched with many different anions. Very commonly, the core of an IL cation is a nitrogenous heterocycle with a quaternized amine. The best known are imidazolium-based IL's, but examples are also known of pyridinium, pyrimidinium, and many others. IL anions also may contain nitrogenous organic compounds, including examples with purines, urea, and others. Due to their physical and chemical behaviors, such as near-zero vapor pressure, tunable viscosity, electrochemistry, gas solubility, and others, IL's are opening broad new areas of industry and technology, such as those disclosed by the inventors in U.S. patent non-provisional application Ser. Nos. 18/749,810 and 18/811,064, both of which are hereby incorporated in their entireties by reference. IL synthesis is currently expensive and generally limited to reactions with petroleum derivatives. IL precursors that are less expensive and that are independent of the petroleum industry would enable many applications that today are not achieved by the IL industry.

Most of the global output of Haber-Bosch ammonia is used to manufacture nitrogen-containing fertilizers for agricultural fields around the world. A significant portion of the crops thus grown are fed to farm animals. Mammals such as pigs and cows excrete most of their nitrogenous waste as urea, which is generally lost as urine. Poultry, however, like all birds, excrete most of their nitrogenous waste as uric acid, a nitrogenous heterocyclic compound with very low solubility in circum-neutral water. Aerobic degradation of purines, including uric acid, is known to produce a variety of possible intermediate compounds of progressively lower enthalpy of formation-ultimately simply ammonia, nitrate, and carbon dioxide. This degradative pathway reflects an energy cascade facilitated by many organisms and a wide variety of facilitating enzymes (Vogels and Van Der Drift, 1976). Uric acid, therefore, represents a high-enthalpy source material for many potential nitrogenous organic compounds, either within the common purine degradation pathway (e.g. allantoin), or closely related derivatives (e.g. caffeine, pyrimidine). In addition to the above examples of sources of nitrogenous waste, many other resources are found in waste products from animals, including those from horses, cows, pigs, poultry, fowl, reptiles, bats, insects, birds, reptiles, and other fauna.

The chemical behavior of uric acid is well known, and methods have been disclosed for its extraction from poultry manure and litter (e.g. U.S. Pat. Nos. 3,860,487; 3,961,096; 4,045,582; 4,196,290). These methods are energy intensive, time consuming, and largely impractical due to problems that are overcome by the novel process disclosed herein by the inventors. Due to these issues, poultry sources have not been widely exploited as industrial sources of nitrogenous compounds. The largest application of these materials is as fertilizer, where most of the nitrogen is lost to the atmosphere or to water runoff. Current methods have been unable to effectively utilize this resource for industry for numerous reasons, including 1) the rapidity with which uric acid biodegrades, through intermediates, to urea, ammonia, nitrate, and carbon dioxide, and the resulting low uric acid concentrations; 2) the very high energy requirements of heating or evaporating large volumes of process water; 3) lack of value added products to support and justify the cost of uric acid extraction methods. The invention disclosed herein addresses all of these issues, and others, by solving numerous problems related to the extraction and refinement of materials from poultry manure and litter, and other nitrogenous waste streams and waste products.

Biochemistry of Poultry Manure and Litter. Fresh poultry manure is a complex matrix composed of numerous carbonaceous organic waste compounds, a nitrogenous component dominated by solid uric acid, an aqueous component containing both dissolved inorganic solutes and organic solutes, and undigested poultry feed containing cellulose, hemicellulose, lipids, and other compounds. The manure typically hosts a robust microbial community that rapidly biodegrades many components of manure, most notably the uric acid component, under aerobic conditions. Uric acid degraders are thought to oxidize uric acid through the enzymatic action of microbial uricase (urate oxidase) and numerous subsequent enzymatic reactions leading to the intermediate formation of allantoin, allantoic acid, ureidoglycine, ureidoglycolic acid, glyoxlyic acid, alloxonic acid, and others, ultimately leading to urea, ammonia, nitrate, and carbon dioxide. (Vogels and Van Der Drift, 1976). This process is enhanced by aeration, and inhibited by anoxia, which limits biodegradation to much slower anaerobic degradation pathways.

The organic fraction of poultry manure contains a diverse array of compounds including proteins, hemicellulose, cellulose, lignin, and lipids. These have formed the basis for a number of disclosed techniques for production of a number of value-added products from chicken manure, including biochar, biocrude, biodiesel, methane, and others (Bora et al., 2020).

The inorganic dissolved solids present in manure are variable, but are dominated by cations K, Mg, Na, Ga and anions PO₄, HCO₃, Cl, SO₄, ranked by molar concentrations. Ammonia and nitrate concentrations can also be considerable depending on the degree of aerobic degradation of uric acid that has occurred, which is often substantial in the case of litters that have been resting before collection. Upon complete evaporation to dryness, resulting salt compositions depend on the relative abundances and thermodynamic solubilities of the various potential salts (Deocampo and Jones, 2014)—these generally include struvite, sylvite, and sodium carbonate (trona or nahcolite) for brines derived from poultry litter fluids.

In addition to the above components, poultry manure typically contains many organisms that are pathogenic to humans. These include E. coli and Salmonella spp. The abundance of pathogens has been shown to decrease with age, such as with composting (Ksheem, 2014). Additionally, compositing will degrade some organic matter, changing its rheology, hydrophilicity, and leading to moisture loss.

Uric Acid. In order to better develop the resource represented by uric acid supplies generated by poultry or other organisms, it is necessary to control the natural degradation pathway that ultimately produces lower-value compounds such as volatile ammonia, nitrate, and carbon dioxide. For example, U.S. Pat. No. 7,674,311 B2 discloses a method of extracting nitrogenous compounds from poultry manure and guano, but it is based on complete oxidation of compounds to volatilize ammonia and generate nitrate, so all intermediate products, which have value as high-enthalpy compounds, are lost. The chemistry of uric acid degradation is complex, affected by numerous factors such as pH, temperature, microbial catabolismand enzymatic activity. Uric acid is a purine compound with three carbonyl groups, that is sensitive to many possible reaction pathways (Golovchinskaya, 1973). Transformations of uric acid have been well studied because of its importance for purine degradation in many organisms generally, and also for its role in human hyperuricemia (gout), which occurs due to pathological uric acid accumulation. Natural uric acid degradation proceeds initially by oxidation, in many cases through different potential intermediates, to form allantoin. Natural uric acid oxidation is generally facilitated by uricase (urate oxidase), a family of enzymes that is produced by a wide variety of organisms, including many bacteria endemic to poultry feces. In poultry litter, subsequent hydrolysis and further degradation of allantoin is carried out by allantoinase-producing organisms. Preservation of allantoin, therefore, or any other desired product in the chain of uric acid degradation, requires halting or altering the sequence of degradation products. This strategy is an important component of the biochemical background to the strategy of the invention disclosed herein.

In addition to uricase, other oxidizing agents are known to oxidize uric acid to form allantoin, including potassium permanganate, ozone, lactose peroxidase-H₂O₂, horseradish peroxidase-H₂O₂, electrolysis, Fenton's Reagent, and others. Most of what is known about uric acid oxidation was learned at the micromolar scale relevant to both hyperuricemia and human protective anti-oxidants, of which uric acid is one of the most important. Depending on the conditions of oxidation, reactants and catalysts present, and other factors, uric acid oxidation may yield a wide variety of products. Chemical modeling by the inventors, however, indicates that reactions may be optimized to maximize the yield of allantoin, and furthermore that it can be scaled up to an industrially relevant scale.

Extraction of uric acid from a complex matrix such as manure, guano, or litter, requires a highly basic (alkaline) solution, as its solubility in neutral or acidic water is very low. Methods of extraction have been disclosed involving the addition of strong base such as sodium hydroxide in sufficient quantity to dissolve large amounts of uric acid, as cited above. Due to the deprotonation of uric acid upon dissolution in basic water, careful monitoring of pH is required to optimize extraction and maintain the desired pH and alkalinity. Beneficiation (concentration) of uric acid from such an extraction fluid for further processing then requires a process to separate the fluid from the extraction matrix, and a step of separating the uric acid from the fluid. A variety of methods are available to separate fluids from the extraction matrix, especially filtration and dewatering methods from the wastewater treatment industry, and industries dealing with solvent processing of agricultural products such as sugarcane, sugar beets, and extracted vegetable oils. Some disclosed processes such as flash heating involve temperatures that are damaging to uric acid or desired products, so care must be taken to maintain the stability of desired phases. Neutralization or acidification of the uric acid-bearing fluid will induce uric acid precipitation, and allow separation and purification. When dealing with volumes of extraction liquids at industrial scale, energy requirements for boiling/evaporating large volumes of water can be prohibitively expensive while potentially increasing the carbon footprint of operations substantially. Energy consumption in uric acid extraction and purification is therefore a major concern, as it has been a major barrier to commercialization in the past. The invention disclosed herein solves this problem in a number of ways, including through the innovative use of mechanical dewatering technologies and reverse osmosis technologies to lower the demand for water in the processing of uric acid, thereby dramatically reducing energy requirements for extraction and purification compared to conventional approaches.

Uric Acid Derivatives. Once separated and purified from a complex matrix, uric acid is a potential reagent and precursor for a wide variety of nitrogenous organic compounds. Reaction of uric acid with other reagents and catalysts to generate desired products may be carried out in a number of possible ways, including in batch reactors, continuous flow reactors, stirring mixing; reactors, mixing annular centrifuges, and others.

One example of such a desired reaction is the oxidation of uric acid to form allantoin. Although this has been accomplished at the micromolar scale in therapeutic and physiological investigations, the invention disclosed herein is a novel industrial application of this process. For reactions carried out in acidic or circum-neutral conditions, which may be necessary to maintain the stability of desired products such as allantoin, the low solubility of uric acid is a major consideration and likely leads to a rate-limiting step in aqueous reactions, according to model calculations by the inventors. One favored approach to carry out aqueous phase reactions with low solubility uric acid is to carry out the reactions in a uric acid saturated solution in contact with, and therefore buffered by, solid phase uric acid. As reactions consume aqueous uric acid, more will then dissolve, allowing the reactions to proceed if other requirements remain met. For example, industrial-scale enzymatic oxidation of uric acid to form allantoin can be carried out in a stirring reactor containing 1) a uric-acid saturated solution; 2) a quantity of solid phase uric acid in contact with the solution; 3) an appropriate quantity of uricase or other enzyme; and 4) other reactants or catalysts. Those skilled in the art will understand that process refinement, optimization, and upscaling benefit from particular enzyme and catalyst selection, enzyme immobilization, and protection of product. Upon reaction completion, product may be further purified by repeated selective dissolution and precipitation using a solvent and conditions depending on the impurities needing removal (e.g. inorganic salts) and the desired level of product purity. The above is only one example, and should not be construed as limiting the invention in any way.

Other high value derivatives of uric acid can be produced through a series of organic chemical reactions that can be optimized depending on the needs of the application. Allantoin, for example, whose derivation by oxidation is described above, contains a core imidazole ring that is itself an important precursor for many compounds, including pharmaceuticals and novel substances such as the family of imidazolium-based ionic liquids. Such compounds can be derived from uric acid by oxidation to allantoin, followed by removal of groups (if desired) using various techniques such as reduction, hydrolysis, exchange, substitution, or others, to yield an imidazole ring that is suitable for established ionic liquid synthesis techniques such as amine quaternization and alkylation to yield a halide salt, that can then undergo anion exchange to produce a desired Ionic Liquid (MacFarlane et al., 2017). Uric acid itself may be used to similarly generate a halid salt, or reacted to produce purine that may then be similarly alkylated. Uric acid may also be reacted with formamide to produce xanthine, or reacted with acetic anhydride and hydrolyzed to produce uracil, either of which may be quaternized and alkylated to generate halide salts and tonic liquids. Uracil can be further reacted to produce pyrimidine, which may be similarly alkylated. The end product halide. salts and ionic liquids of all these reaction pathways, as well as the intermediates therein, and others not described, are valuable nitrogenous organic compounds that can be produced without petroleum-based precursors using the invention disclosed herein.

To the degree that urea is produced by the degradation of uric acid and other nitrogenous organic compounds, it also represents both a valuable resource itself, and a potential precursor of other value added products. These include uracil, which can be synthesized by the reaction of urea with malic acid or acetylene dicarboxylic acid (or other dicarboxylic acids). Uracil can be further reacted through reduction or hydrolysis to produce pyrimidine. Urea, uracil, and pyrimidine can all be alkylated, for example through amine quaternization to produce a halide salt, which can be a precursor to ionic liquids.

Example. Following is a model of an example of the extraction and purification of several value added products from a poultry litter waste stream, embodying, in part, the invention disclosed herein. This example should not be construed as limiting the invention in any way, but rather it is to exemplify some conditions and controlling factors of product generation. The example poultry litter stream enters the system, shown schematically in FIG. 1, with a composition typical for broiler litters, as reported by Chastain et al. (1973) for moisture, organic and nitrogenous compounds, and Ksheem (2014) for soluble salts. In this example, this amounts to 21.5% moisture, 78.5% solids, 27 kg/ton organic nitrogen, 46.7 mg/g K, 1.1 mg/g Ca, 5.6 mg/g Mg, 3.8 mg/g Na, 7.4 mg/g Cl, 13.3 mg/g PO₄, and 0.3 mg/g SO₄ (all weights with respect to dry mass). If only 50% of the organic nitrogen is held in uric acid molecules (the rest having been lost to degradation), then that corresponds to about 40 kg of uric acid per ton of litter. In the water wash, a volume of water sufficient to dissolve the soluble salts is mixed with the litter, and then separated into a liquid stream containing soluble salts, and a solid stream containing the insoluble residue. The liquid stream is then dewatered, precipitating the solid salts. Thermodynamic calculations of salt stabilities indicate that llkg of struvite, 12 kg of sylvite, and 15 kg of nahcolite will precipitate, per ton of initial wet litter. The solid stream then enters an alkaline wash where it is mixed with a basic, alkaline fluid of sufficiently high pH, over 8, or over 9, or most preferably over 10, in order to dissolve the solid uric acid contained in the solid stream. Strong base addition is carried out sufficient to neutralize and exceed the acidity generated by uric acid dissolution, thus requiring monitoring of solution pH. The alkaline wash is then separated into a liquid stream, containing dissolved uric acid, and a solid stream containing insoluble residue, mostly organic matter.

The liquid stream is then neutralized or acidified by mixing with a sufficient quantity of strong acid, thus driving down the pH and causing uric acid to precipitate. The solid stream is then separated as a product. The neutral or acid solution, containing dispersed solid uric acid, is then placed into an oxidation reactor, in which it is converted to allantoin. The solution is then dewatered to separate out the product allantoin, amounting to 37.3 kg in this example. The above process description is intended as a general overview, and should not be construed as a comprehensive description of any particular process, which may require particular controls, repeated steps, addition or removal of material, phase separations, purifications, or other optimizations.

Volume Reduction and Dewatering. Many material processing approaches such as those described above involve volumes of solvent, in many cases water, that are crucial for some extraction or separation steps, but that must subsequently be reduced or eliminated to concentrate desired products. Such volume reduction, or even complete dewatering, can be accomplished in a number of ways, with many possible approaches, all with varying capital costs, energy requirements, maintenance and consumables, impacts on reagents and products, and efficiencies.

The first major challenge to volume reduction is the abundance of highly hydrophilic solid organic matter. In particular, Extracellular Polymeric Substances (EPS) are a known component of animal manure that increases the hydrophilicity of the solid matter, increasing its ability to retain water. In organic-rich matrices such as sewage sludge or animal manure, water can be described as being in one of three forms-free water, interstitial water, and bound water (Cao et al., 2020). Free water is not directly bound to solids, and hence is most easily separated from the matrix. Interstitial water resides between sludge particles, and is in close contact with the surfaces of particles-hence they are subject to surface tension interactions, and are much more difficult to separate from the matrix. Bound water occurs as waters of hydration in close association with EPS (largely composed of proteins and polysaccharides), or as intracellular waters. Separating bound water requires disruption of EPS hydration bonds and/or ruptuting cell membranes, both being energy demanding processes,

The simplest method of volume reduction is drying. Many methods of drying are available, including drum drying, fluid bed drying, flash drying, and many others. Flash drying is a preferred method in a number of disclosed methods of uric acid extraction (e.g. U.S. Pat. No. 4,196,290). None of these are likely to-find widespread industrial application, however, because of the inescapable requirement of 10^{6.35} J/kg, the enthalpy of vaporization of water—this makes rapid evaporative drying impractical for industrial processes that cannot support such large expenditures of energy. Evaporation pools are an alternative approach, when time and space allows, in cases where large surface areas can be exposed to dry air, in cases when evaporation rate exceeds process water inflow rate, and equilibrium vapor pressure depression due to elevated salinity is accommodated (Bohren and Albrecht, 1998).

Mechanical methods of dewatering are common in the fields of wastewater treatment. They include many possible configurations, and generally fall into two categories—centrifuges and filter presses. Filter presses include designs such as belt presses, screw presses, fan presses, frame presses, and others. Centrifuges may involve radial separation of solids from liquids, and may also involve membranes or filters within the configuration. Mechanical methods of dewatering also include pretreatment of inflow, such as chemical oxidation, biological digestion, ultrasonication, or pretreatment with flocculants or coagulants. All of these approaches are effective at separating free water, but interstitial and bound water often remain recalcitrant (Cao et al., 2020).

Several membrane-based approaches can be used in industrial settings to separate solute streams into dilute and concentrated streams, Reverse osmosis (RO) uses mechanical pressure to force water through a semipermeable membrane that retains solutes in a residual brine. Typical commercially available RO's for industrial applications can produce a stream of dilute water that is then available for reuse, while concentrating salts into a brine of a maximum of about 70,000 mg/L (Davenport et al., 2018). To produce such a brine, an osmotic pressure of about 10^{6.77} Pa must be maintained across the membrane, leading to the energy cost of operation. High pressure RO systems are theoretically possible, and in development, but are not currently in widespread industrial use,

A number of other approaches may be considered, including electrodialysis, which uses electricity to assist in the migration of ions to an ion exchanging, semipermeable membrane, and mechanical vapor compression, which efficiently vaporizes water. These have high energy consumption requirements, although under some circumstances may be appropriate for water reuse and brine concentration applications.

Membrane based approaches to volume reduction all require significant pretreatment of feed streams to prevent fouling by particulates or biofouling by micro-organisms. These may include physical treatments such as filtration, microfiltration, or ultra-filtration. They may also include chemical treatments such as chlorine, ozone, other oxidants, or anti-microbial chemicals.

Depending on the application, the above methods may be used singly or multiply, in series, or in repeated cycles, or in different Segments of the overall process, to achieve the desired streams of concentrated products, dispose of waste, and maximize efficient reuse of resources,

Industrial Enzymatic Reactors. Among the several options available for uric acid oxidation, enzymatic treatment offers several factors that make it innovative and valuable for inclusion in the methods disclosed herein. First, enzymatically catalyzed uric acid oxidation can be carried out at temperatures that are practical to maintain and favorable for the stability of reactants and products (i.e, between 10-70° C.). Second, the reaction kinetics of consistently produced industrial enzymes can be well characterized and controlled, therefore reaction processes can be well controlled and understood. Additionally, if efficiently and effectively immobilized, enzymes can be reused many times, reducing process costs. As understood by those with skill in the art, and as described by Weetall and Pitcher (1986), several major factors must be considered in the design and optimization of industrial-scale enzymatic reactors. These include the source and purity of the enzyme, the carrier/support structure to which the enzyme is attached, and hence, its chemical behavior, the method of attachment, the type of reactor, and operating strategies. Some examples of possible choices available for uric acid oxidation are shown in Table 1-optimization of these choices will depend on the particulars of specific applications.

TABLE 1
Some selected examples of choices in optimization of industrial
immobilized enzymatic uric acid oxidation. This list
is only exemplary, is not exhaustive, and is not to be
construed as limiting the invention in any way.

| Enzyme | Enzyme | Enzyme | Attachment |
|---|---|---|---|
| (Source) | Purity | Carrier | Method |
| Uricase | Bulk Cellular | Nylon | Ionic sorption |
| (Candida sp.) | Lysate | | |
| Uricase | Purified | Zeolite | Gluteraldehyde |
| (E. coli) | Extract | | Cross-Linking |
| Horseradish | | High-Amylose | Matrix Entrapment |
| Peroxidase | | Starch | |
| (Armoracia | | | |
| rusticana) | | | |
| | | Sepharose | EDAC Coupling |
| | | | Woodward Reagent |
| | | | K Coupling |

Biocrude and Biogas Generation. The organic matter remaining following the intensive washes necessary for extraction of salts and uric acid is a residue that is still rich in complex organic molecules. These are, principally, lignins, cellulose, hemicellulose, lipids, and proteins. Following the removal of most nitrogenous species, these materials are well suited for treatment for the generation of valuable materials such as biogas, biocrude, and other carbon-rich compounds. Methods are well known for the methanogenic generation of biogas, generally by anaerobic digestion (Manogarau et al., 2022). Hydrothermal liquifaction of chicken manure has been shown to produce a range of compounds including solid biochar and liquid biocrude with high concentrations of long chain (C16-C27) acids and esters (Lu et al., 2018). Although these are larger molecules than those typically in fuels such as gasoline or diesel, they are a potential feedstock for hydrocracking or other methods of generating valuable petroleum hydrocarbons. Furthermore they are suitable precursors for many potential compounds that can serve as substitutes for products currently derived solely from petroleum, such as petroleum jelly, petroleum lubricants, fuels, and other materials derived from long chain compounds.

### SUMMARY

The novel system described herein comprises a system and method for the processing of animal manure, litter, or similar waste products to extract various substances for the creation of desired molecular compounds and for the refinement and optimization of the chemical and physical properties of the waste stream for use as a fertilizer.

The first part of the system utilizes aqueous phase beneficiation of the waste stream through a series of sequential solubility extractions to separate salts and other desired dissolved solids. After removal of the salts and other dissolved solids, a selective alkaline solubility extraction is used to remove uric acid from the waste product. The solubility extractions each involve a grinding, mixing, and/or blending of the organic matter with water or an alkaline solution to dissolve the desired compounds for extraction, followed by the mechanical dewatering of the waste product solution. Once dewatered, the waste product is mainly comprised of wet organic matter that is then removed for composting or fertilizer production. The first aqueous phase extraction from the waste product primarily contains various soluble salts, which are further concentrated through a reverse osmosis system to produce a fresh water stream for reuse and a salt brine. This first aqueous phase extraction may be performed one or more times in sequence as necessary to remove all of the desired salts from the waste product prior to the second aqueous phase extraction. In the second aqueous phase extraction a basic solution is used to extract uric acid from the waste stream. After the dewatering process removes the uric acid dissolved in the basic solution, the organic matter and other residual compounds left in the waste product move on to a separate organic matter derivatives processing system. The basic solution containing dissolved uric acid is then combined with an acid compound to achieve a circum-neutral pH, effectuating the precipitation of uric acid from the solution. Upon precipitation, the uric acid is filtered or otherwise extracted from the solution. After extraction the uric acid can be processed to allantoin or other uric acid derivative by way of methods described infra.

### DESCRIPTION OF THE DRAWING

FIG. 1 is a flow chart of the aqueous phase beneficiation process to separate components of an animal waste product for the creation of various value added products.

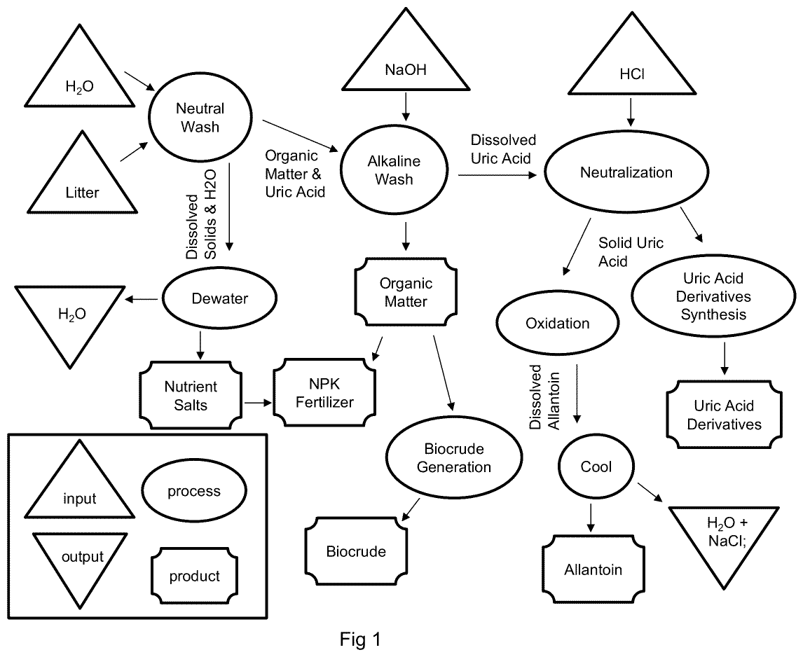

### DETAILED DESCRIPTION

The process begins with the grinding, shredding, or other disaggregation of an animal waste product. The waste product is then mixed with water to dissolve the soluble salts and other soluble compounds contained therein. Dissolution may be enhanced by mixing, stirring, or otherwise agitating the solution in a mixing vat, stirring and scraping container, or similar. Once dissolved, the salt-bearing solution is separated from the solid phase of the waste product by use of one of several different dewatering devices or methods. These devices include all of those mentioned supra in the scientific background and any others that may be suitable for the task of dewatering a specific animal waste product. During the dewatering process regardless of what method is used for dewatering, both the outgoing solid phase and the outgoing aqueous phase must be collected for further processing as they exit the dewatering device.

Upon exiting the dewatering device, the salt-bearing aqueous phase is filtered to remove particulates that passed through the dewatering process with the aqueous phase. This filtration may be graduated with a series of progressively smaller filters, ranging from millimeter or coarser, to micrometer or nanometer scale, or it may be single step filtration process at an optimized size range. Once filtered, the salt-bearing aqueous phase solution will be concentrated into a brine or otherwise processed in a way that allows for the eventual collection of the dissolved salts. In some embodiments, the collection and use of the salts may require only concentration into a brine. Others may require or be benefitted by precipitation of the dissolved salts. There are many common methods of creating concentrated brines and harvesting salts known to those skilled in the art, and all of these various methods are contemplated by the inventors as potential components of the system described in this disclosure. One of these methods contemplated by the inventors is the use of reverse osmosis systems to create a concentrated brine and a stream of purified water for reuse in the wash cycle. In this non-limiting example, the concentrated brine created by the reverse osmosis system may be further concentrated by an electrodialysis system, a mechanical vapor compression system, or evaporation, whether induced or natural such as heating or evaporative brine ponds.

Due to the inefficiency of dewatering technology, the outgoing solid phase from the dewatering process will still contain roughly 50-80% moisture, including a percentage of the dissolved salts for extraction. In order to extract the highest percentage of the salts possible, this process can be repeated two or more times in sequence.

Once the salts have been removed from the animal waste product, the solid phase of the waste product still contains uric acid, which is insoluble in water at circum-neutral pH. In order to remove the uric acid, an aqueous phase beneficiation using a basic solution is used. To begin this process, the solid phase of the organic waste product must be disaggregated after the previous dewatering step. This step may be accomplished through similar means as those used for the previous wash cycle, such as grinding, shredding, mixing, etc., with the addition of fresh water. The water may be basified before being added to the mixture, after the mixture has been created, or during the mixing process by the addition of a basifying agent, such as NaOH. Once the pH of the mixture has been raised, the mixing process should continue until substantially all of the uric acid has been dissolved into the basic aqueous phase.

After dissolution, the mixture is dewatered by a similar dewatering method as those that may be used in the salt wash process described supra. This allows for the separation and collection of the basic solution containing the dissolved uric acid from the solid phase of the waste product, though this wash and dewatering process may be repeated multiple times in order to increase the percentage of uric acid extracted from the waste product,

After the uric acid bearing basic aqueous solution has been removed, the solution is brought to a circum-neutral pH, or a lower pH, in order to precipitate the uric acid. Once precipitated, the solution can be filtered to remove the uric acid. This filtration can be accomplished by a number of different methods known to those skilled in the art, all of which are contemplated by the inventors and included as part of the system described herein, though a standard micron-sized mechanical or physical filtration will usually be sufficient to remove the uric acid. The aqueous phase may then be recirculated for use in a subsequent wash cycle. The purity of the solid phase uric acid may be enhanced in a number of ways, including repetition of the basic aqueous phase beneficiation, and/or washing or rinsing with purified water, ethyl alcohol, methyl alcohol, ethyl ether, or other solvent.

The three outputs from the aqueous phase beneficiations and sequential solubility extractions are 1) the salts which have been removed into the concentrated brine; 2) the remaining solid phase of the initial animal waste product, which is mainly comprised of lignocellulosic biomass and other organic biomass; and 3) the solid uric acid.

Each of these resulting outputs can be further processed to create valuable products for commercial, agricultural, industrial, and consumer products. The following are several non-limiting examples of the products envisioned by the inventors. These descriptions comprise a non-exhaustive, non-exclusive list of possibilities. The inventors' intention is to preserve the right to all products created through the use of the aqueous phase beneficiation of animal waste products, even if not explicitly described herein.

In some embodiments, the salts and the solid phase of the waste product may be recombined after the removal of uric acid to form a fertilizer, potting soil, or similar commercial or agricultural product. The solid phase may be composted, flash dried, or otherwise treated to prepare it for processing and recombination with the salts. The salts may be added in either a solid or a liquid for depending on the desired moisture contents, salt contents, physical properties of the resulting fertilizer, and energetic considerations of fully or partially evaporating the salt brine. This process allows for the selective creation of organic fertilizers from animal waste products with customizable nutrient content. Once combined into the desired proportions, the fertilizer may be pelleted or similarly processed by any number of standard methods for commercial fertilizer production.

In some embodiments, the uric acid may be further processed into allantoin. This may be accomplished through oxidation of uric acid by a variety of different means, including but not limited to, exposure to ozone, enzymatic digestion, UV photo-oxidation, high energy particles, radiation, oxidizing chemicals such as potassium permanganate, and many others known to those skilled in the art. While the inventors preserve claims to all potential methods of deriving allantoin from an animal waste product, the two main embodiments envisioned by the inventors as most readily available for commercial production of allantoin are oxidation through ozone exposure and enzymatic reaction using a variety of different possible enzymes.

The use of ozone to oxidize uric acid may be accomplished through the use of an industrial or commercial ozone generator to create ozone gas which 1 s then brought into contact with the uric acid in several potential different ways known to those skilled in the art, The most efficient way to accomplish this is to mix the uric acid with an aqueous solution and cause the dissolution of ozone into the solution, such as through a bubble column or similar device in which an atmosphere containing ozone is brought into contact with a liquid containing uric acid, whether solid or dissolved.

In other embodiments an enzyme may be used to convert uric acid into allantoin through oxidation, as described in the scientific background. This process involves the use of enzymatic reactor technology that has been disclosed for use in many other industrial and commercial processes. In some embodiments the appropriate enzyme may be immobilized onto a surface for interaction with uric acid as a substrate in either a stirring reactor or a packed bed reactor, or other type of reactor configuration. In other embodiments, the enzyme may be added to the uric acid mixture without immobilization or may be actively created through microbiological processes for continual conversion of uric acid to allantoin. In some embodiments, a reagent such as H₂O₂ may be added to the uric acid solution to facilitate the enzymatic oxidation to allantoin. The inventors envision the possible use of all currently known and disclosed methods of enzymatic reaction as a component of the novel system and method claimed by the inventors in this disclosure.

Regardless of the method by which the allantoin is created, the resulting solution must be processed to collect the allantoin, refine and purify it for commercial sale or use. Depending on the volume of the aqueous solution containing the allantoin, along with the pH and temperature, the allantoin may be dissolved in the solution, mostly precipitated from the solution, or a mixture of both dissolved and precipitated phases. There are multiple ways to fully precipitate the allantoin for collection that are known to those skilled in the art. These include evaporation, lowering solution temperature, altering the solution pH, lyophylizing the solution, and other potential methods known to those skilled in the art, all of which are reserved by the inventors as potential components of various embodiments of the system and method claimed here. In some embodiments, a reverse osmosis system may be used to aid in the concentration of allantoin in the solution and precipitation therefrom. After collection, the allantoin may be subjected to a series of washes and filtrations to achieve the required purity for commercial use. A final step in this process may be to lyophylize the allantoin in some embodiments.

In some embodiments other chemical compounds may be produced from the uric acid, the organic matter contained within the waste product, or a combination of the two. Examples of potential compounds produced using uric acid as a precursor include, but are not limited to, purines like caffeine, xanthine, theobromine, nucleosides and nucleotides of adenine and guanine, theophylline, allopurinol, adenosine triphosphate, and other purines and purine derivatives, such as complex sugars and alloxan. Other potential derivatives of uric acid include other purines, pyrimidines, and imidazoles, such as murexide, parabanic acid, ureidohydantoin, nucleosides and nucleotides of cytosine, thymine, and uracil, histadines, oxazoles, thiozoles, and others known to those skilled in the art of organic chemistry synthesis. Examples of compounds that may be created from the organic matter include biochar, biocrude, and biocrude derivatives, such as petroleum jelly, oils, lubricants, fuels, solvents, personal care products, cleaners, polymers, plastics, and other petroleum-based product substitutes. Examples of compounds that can be made as a result of combining the organic matter derivatives with the uric acid derivatives include ionic liquids and ionic liquid precursors, such as cations based on imidazolium or pyrimidinium bases and anions, and other complex or simple organic molecules including, e.g., aliphatics, aromatics, resins, and ashpaltines.

What is claimed:

1. A method for processing and differentiating an animal waste product into separate end products comprising the steps of:
Disaggregating the animal waste product using means for disaggregation to create a disaggregated animal waste product;
Mixing the disaggregated animal waste product with water to create a mixture containing a solid phase of the animal waste product and an aqueous phase having dissolved within said aqueous phase any water soluble substances contained within the disaggregated animal waste product;
Separating the solid phase of the animal waste product from the aqueous phase containing the dissolved water soluble substances by means for dewatering;
Filtering the aqueous phase to remove any particulate matter contained therein;
Creating a concentrated brine from the aqueous phase containing the dissolved water soluble substances by means for brine creation;
Disaggregating the dewatered solid phase of the animal waste product by means for disaggregation;
Mixing the disaggregated and dewatered solid phase of the animal waste product with an alkaline aqueous solution having a pH greater than 8 to dissolve the uric acid and derivatives thereof contained within said solid phase of the animal waste product;
Separating the solid phase of the animal waste product from the alkaline aqueous solution containing dissolved uric acid derivatives thereof by means for dewatering.

2. The method of claim 1 further comprising the steps of:
Precipitating and collecting the uric acid and derivatives thereof from the alkaline aqueous solution by lowering the pH of said alkaline aqueous solution to below 9;
Oxidizing the uric acid by means for uric acid oxidation to convert the uric acid to allantoin.

3. The method of claim 1 further comprising the steps of:
Precipitating and collecting the uric acid and derivatives thereof from the alkaline aqueous solution by lowering the pH of said alkaline aqueous solution to below 9;
Processing the uric acid to create derivatives thereof selected from the group consisting of: purines, urea, caffeine, xanthine, theobromine, nucleosides and nucleotides of adenine and guanine, theophylline, allopurinol, adenosine triphosphate, complex sugars, alloxan, pyrimidines, imidazoles, murexide, parabanic acid, ureidohydantoin, nucleosides and nucleotides of cytosine, thymine, uracil, histadines, oxazoles, thiozoles.

4. The method of claim 1 further comprising the steps of:
Precipitating salts contained within the brine by means for precipitating salts;
Collecting the salts for recombination with the solid phase of the animal waste product to produce a fertilizer product.

5. The method of claim 1 further comprising the steps of:
Processing the solid phase of the animal waste product to create a lignocellulosic biomass derivative selected from the group consisting of: textiles, paper products, biochar, biocrude, biocrude derivaties, petroleum jelly, oils, lubricants, fuels, solvents, personal care products, cleaners, polymers, plastics, and petroleum-based product substitutes.

6. The method of claim 1 further comprising the steps of:
Using derivatives of the solid phase of the animal waste product in combination with derivatives of the uric acid to create a product selected from the group consisting of: ionic liquids, ionic liquid precursors, imidazolium cations, pyridinium cations, imidazolium bases, pyridinium bases, imidazolium anions, pyridinium anions, aliphatics, aromatics, resins, and asphaltenes.
